(19) United States
(12) Patent Application Publication
(10) Pub. No.: US 2011/0112574 A1
(43) Pub. Date: May 12, 2011

(54) DEVICE FOR MANUAL TRACTION WOUND CLOSURE

(76) Inventor: Pal (Paul) Svedman, Malmo (SE)

(21) Appl. No.: 13/008,942

(22) Filed: Jan. 19, 2011

Related U.S. Application Data
(63) Continuation-in-part of application No. 12/557,868, filed on Sep. 11, 2009.

(51) Int. Cl. A61B 17/08

(52) U.S. Cl. 606/216

(57) ABSTRACT

A traction device includes an advancing member equipped with a zone through which a member is forcibly advanced, a pair of cooperating flexible members, wherein each flexible member has at least one side thereof at least partially coated with an adhesive material, wherein the adhesive material of each the member is connected to opposing skin surrounding a wound such that a remaining portion of each member extends over and above the wound and wherein the remaining portions are connected through the zone of the advancing member in a manner to advance the remaining portions and draw the opposing surrounding skin toward each other in a manner to aid in closure of the wound.

This is a continuation in part of U.S. Ser. No. 12/557,868 filed Sep. 11, 2009.

### FIELD OF THE INVENTION

The instant invention relates to wound healing and more particularly, but not by way of limitation, devices and methods for stretching and expanding adjacent tissues medially to cover a wound.

### BACKGROUND

Open clinically significant wounds heal by contraction, a slow process which includes scar contraction and re-epithelialization and leaves scar and usually functional and aesthetic deformity. Disfigurement and functional disturbance is frequent. Surgical treatment, which can prevent, break or ameliorate the course, may be delayed or contraindicated for various reasons, or may just not be an available option. As a consequence, deformity and scarring are problems to be addressed.

Mechanical forces play an important part for tissue development and function through effects induced on the cellular and extracellular level. When traction is applied protractedly to the skin from a surgically implanted outwardly expanding balloon (i.e. a tissue expander), an area of the overlying skin and subcutis increases by formation of new tissues, and can be used surgically for covering an adjacent open wound. During the expansion, the epidermal thickness, i.e. the number of cells, increases, and that of dermis and subcutis decreases. Blood flow in the expanded tissue increases, and sensitivity remains intact. When skin traction is performed acutely, markedly higher stretching forces have to be applied, and the degree of skin lengthening is the result of mechanical stretching rather than formation of new skin.

Surgical devices have been disclosed which use skin expansion or acute stretching to close open wounds of surgical or non-surgical etiology. Technically, pins or hooks are fastened near the wound's edge at opposing sides, usually through several fixture points in the fibrous dermal layer of the skin. Medial traction is accomplished by means of sutures, rubber bands, plastic straps or screws. By these means, skin is expanded or stretched medially until wound closure can be achieved.

Disadvantageously, the traction force becomes reduced over time if the sutures of bands are not readjusted at intervals. Also, access for effectively treating the underlying wound becomes restricted. Neither do such approaches allow centripetal traction nor predetermined variation of traction to maximize tissue gain.

In abdominal compartment syndrome of differing etiology increased intraabdominal pressure requires pressure decompression. The abdomen is opened by means of a long medial incision through the anterior abdominal wall which includes fascia and peritoneum. This makes the abdominal wall including fascia contract laterally to such a degree that surgical closure can be accomplished only after the tissue has been stretched stepwise until its normal width has been regained. Technically, this is accomplished by suturing a nonyielding perforated polymer mesh circumferentially to the exposed edges of the abdominal wall fascia under traction, and repeating the procedure at 2 to 3 day intervals until the abdominal wall length has been regained. At this point, the mesh is removed and the wound resutured in layers. Throughout, the wound is usually exposed to negative pressure treatment, which also allows removal of excess abdominal fluid contributing to the distension.

The rate of formation of new tissue relates to the magnitude and duration of the applied traction force, and to the width of tissue (skin) exposed to traction of a given magnitude. The relationship between these parameters has not been evaluated systematically, but loading an undefined width with 0.26 kg (2.5N or 190 mmHg) to 3 kg (29N or 760 mmHg) is reportedly a reproducible means to produce such skin expansion in infants and adult patients respectively. Using broad straps adhered to the skin, this range of loading is used also to disengage fractures or dislocations. To lengthen skin acutely, higher weights are required.

Treatment of wounds by means of negative pressure applied through a contractile open cell foam dressing includes tissue traction. Both the wound and the adjacent skin become exposed to medial pull as the dressing contracts during application of suction. The degree of pull is limited by friction between the foam cells and onlying polymer film, between onlying cells, and between said cells and wound bed. A limitation is that the level of negative pressure cannot be increased beyond the upper pressure range recommended for wound treatment.

### SUMMARY OF THE INVENTION

It is a general object to improve wound healing.

It is a further object to provide a device for wound healing.

A further object is to provide a device and method for improved traction in treating wound healing.

Another object is to improve the treatment of open major wounds to prevent disfigurement and functional disturbance.

Accordingly, one embodiment is directed to a traction device which includes an advancing member equipped with a zone through which a member is forcibly advanced, a pair of cooperating flexible members, wherein each flexible member has at least one side thereof at least partially coated with an adhesive material, wherein the adhesive material of each the member is connected to opposing skin surrounding a wound such that a remaining portion of each member extends over and above the wound and wherein the remaining portions are connected through the zone of the advancing member in a manner to advance the remaining portions and draw the opposing surrounding skin toward each other in a manner to aid in closure of the wound. Another aspect is directed to a method for applying traction to tissues adjacent a wound, which includes the steps of:
(a) positioning a contractible actuator adjacent a wound, wherein said actuator has portions adhesively connected to surrounding skin of the wound; and
(b) actuating the actuator to draw said portions medially inwardly to effect closure of the wound.

Such outcomes may be counteracted by means of the present invention, which by providing new means and methods for elongating tissue in a controlled way allows intact skin and subcutaneous tissue to be moved medially to markedly reduce the size of the wound, and eventually achieve nonsurgical wound closure with innervated composite tissue in kind. As for the present invention, the primary field of use is for achieving wound closure by protracted traction, although closure through acute traction may also be feasible. In either application, the distribution of traction force evenly at the skin surface level (rather than concentrated to points of dermal penetration) prevents local tissue compression and may thus avoid skin necrosis. The invention may also be used in patients with open abdomen to stretch contracted abdominal wall.

Use of the invention may typically be indicated 1) when reconstructive surgery cannot be undertaken because of the patient's condition, 2) when for various reasons such surgery has to be delayed, and 3) when surgical treatment is unavailable. Its use is contraindicated when the wound or the adjacent skin is damaged or infected, and when the skin has poor blood circulation or is inflicted by disease engaging the dermoepidermal junction.

In an embodiment, traction to the skin adjacent to a wound is accomplished according to the invention by means of an actuator placed over the wound and adhered to the surrounding skin by an adhesively applied flange which may be circular or divided into two parts extending laterally. The actuator interior consists of elastic, compressible open cell material, and is airtightly connected to a suction pump by means of a tube according to the state of the art. When exposed to negative pressure, the actuator contracts medially and the force created by suction exerts medial traction on the flange(s) and underlying skin and/or tissue. The maximal pressure range may extend from 50 mmHg to 760 mmHg below atmospheric pressure, with clinical range between 100 mmHg and 650 mmHg below atmospheric pressure. The amount of medial pull is dependent upon the level of the applied negative pressure, the compressibility of the cell material and the resistance to traction offered by the tissues underlying the flange(s). Under in vitro conditions, the medial contraction of an actuator has reached 90 per cent. The adhesive flange(s) may be inelastic or elastic and yielding to stretch to an extent that variably accommodates the increase in length as the underlying skin becomes stretched medially. When the flange is elastic, the traction force may become distributed over a relatively wide area of the adhered skin, minimizing the risk of tangential shear within the skin. The skin flange(s) may or may not be fitted with an inner, circumferential rim free from adhesive, and should preferably be transparent to allow visual assessment of the underlying skin with respect to viability. The flange film may finally contain pores which allow evaporation of water, which may reduce skin adhesiveness. Clinically, the devices according to the invention may be changed from every three to six days.

The undersurface of the adhered actuator may cover the wound only partially, leaving access for applying a wound dressing from the sides. This dressing may include active substances distributed in fiber, open cell or gel material, or it may constitute an occlusively applied typically open cell, pore or fiber dressing with access port(s) used for negative pressure wound treatment, eventually combined with fluid supply. To provide access also when the device according to the invention blocks access to the wound, the actuator may be fitted with at least one conduit, typically extending between its exterior and interior side. The conduit allows room for at least one flexible tube. As described above, the tube(s) can for instance accommodate negative pressure wound treatment with or without concomitant supply of treatment fluid to an underlying open cell pore or fiber dressing according to the state of the art.

The actuator according to the invention may be used as roof in a wound treatment chamber during ongoing actuator traction. In this application the external aperture of said actuator conduit is fitted with resealable port means. Such treatment may for instance include delivery of analgesics and antibiotics. The chamber may furthermore be used as growth chamber and supplied with growth factors, growth media, genes and cells, including stem cells and fetal cells. In both applications, the chamber may contain a biological or synthetic matrix acting as a scaffold for cell growth.

The pump used for compressing the actuator by suction can preferably include a gauge for measuring negative pressure, a mechanism to adjust and maintain the pressure according to clinical demand, and an alarm to warn of air leak in the system, wherein all components can be state of the art. The pump may be controlled by computer technology, eventually by telemetry or manually. The pumps may be portable. A manual or syringe pump fitted with manometer may be used under special circumstances.

The controlled continuous, intermittent or cyclic application of skin traction according to the invention may enhance blood flow and stimulate tissue expansion, applications based on known regenerative effects on cell and tissue function, and may find use for testing a range of stretch stimuli with regard to further regenerative effects.

When suction therapy is administered through an underlying occlusively applied open cell, pore or fiber dressing, a preferred option may be to use a double suction pump connected via separate tubes to actuator and dressing respectively. Suction may be applied to actuator and underlying dressing simultaneously, or to the dressing only after a slight delay. The reason for the latter is that activation of the actuator is expected to constitute the main wound contracting force. The combined negative pressure of actuator and dressing should remain well below that causing stretch-related damage of the adjacent skin.

In a special application, the invention is used in connection with open abdomen, i.e. extensive wounds of different etiology penetrating through the thickness of the abdominal wall. In this application the device according to the invention is used within the wound. The flange of the device is typically connected by suture to the abdominal fascia edges rather than adhered to the skin surface. The flange consists of a perforated mesh whose pores permit excess abdominal fluid to be drained from the underlying abdominal cavity.

One embodiment of the invention is directed to a device for applying traction to tissues adjacent to a wound. The device includes (a) a contractible actuator to be positioned within a wound; (b) a cover, which can be flexible, configured to enclose the actuator and configured to maintain reduced pressure and degree of contraction therein; (c) a flange extending laterally from an actuator base; and (d) a reduced pressure device for connection to a source of suction, the reduced pressure device cooperating with the cover to supply the reduced pressure within the actuator. The actuator can be contracted by suction cooperating with the flange to produce medial traction of adjacent fascia and abdominal wall tissues, and reduce the size of the wound. By virtue of the instant invention, there is less chance for deformity and scarring to occur.

A further embodiment of the invention using adhesively applied flanges to achieve medial traction and elongate the skin to reduce the size of a wound, or eliminate a wound, is described in this application in part. The basis of this later embodiment is to stretch and expand such skin medially, either with manual tools, and to repeat such medialisation procedures at intervals of 1-4 days according to progress in skin stretching. There is also a place for combining these mechanical techniques with suction-dependent traction when the degree of pull on the tissues needs to be increased.

### BRIEF DESCRIPTION OF THE DRAWINGS

FIG. 1 shows a schematic perspective clinical view of a device for transverse mechanical traction by means of bi-layered adhesive sheet flanges according to one embodiment of the invention.

FIG. 2 shows a partial view of FIG. 1 accomplishing traction by use of single layer adhesive sheets.

FIG. 3 shows a schematic cross-section of an embodiment of a grip used for vertical pull.

FIG. 4 is a grip as in FIG. 3 with addition of a traction force gauge with handle.

FIG. 5 shows a cross-sectional partial view of a hinged double roller device with handle compressing adhesive sheets according to the invention.

FIG. 6 shows a schematic perspective view of a traction device activated manually by a screw and lever mechanism according to the invention.

FIG. 7 shows a schematic perspective view of a technique in which upwards protruding adhered flanges can be exposed serially to manual traction.

FIG. 8A shows a schematic perspective clinical view. The activating device has been removed, and only narrow vertical flanges retained. The forces applied to horizontal flanges and skin at each side of the wound are indicated.

FIG. 8B shows a similar view where medial traction has been increased by an onlying actuator operated by negative pressure.

### DETAILED DESCRIPTION

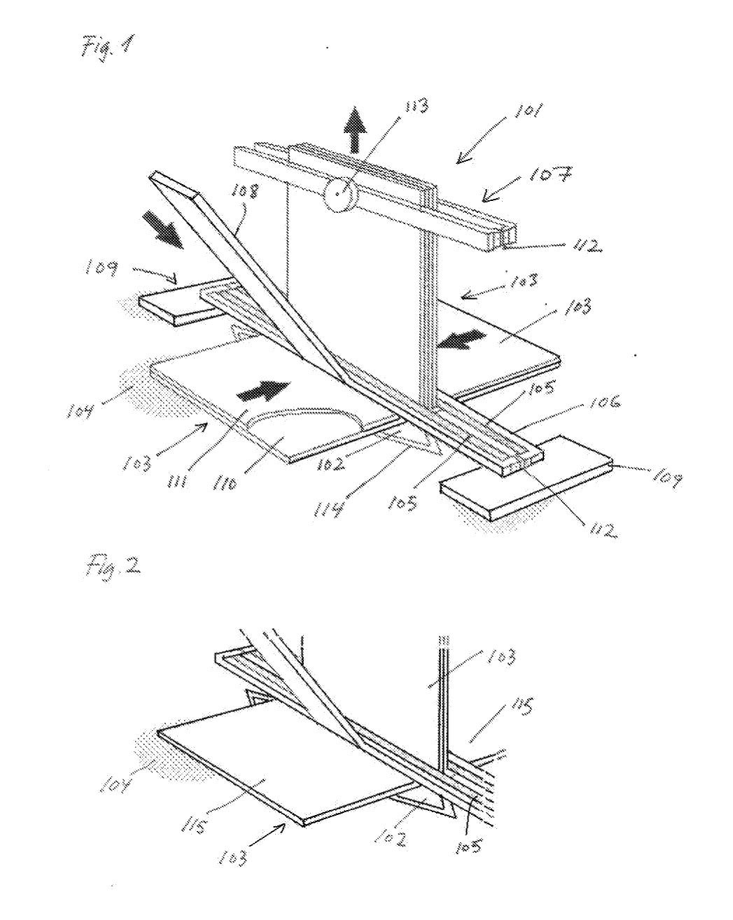

Referring now to the drawings, FIG. 1 shows a schematic perspective clinical view exemplifying a manually operated traction device 101 for closing wound 102 by traction according to the invention. Device 101 includes in this example a flexible preferably vapor permeable adhesive film layer 110 in direct contact with the skin and top layer 111, directionally stable eventually thicker. Layer 110 may suitably consist of vapor permeable polyester, polyurethane or latex with adhesive according to the state of the art. Top layer 111 is typically less adhesively connected with the underlying layer 110 and may consist either of cellulose or polymer.

The layers 10 and 11 form flanges 103 wherein at least part thereof are in adhesive contact with the skin 104 on each side of the wound 102. The flanges 103 extend medially over the wound 102, meet the counterpart from the opposite side between rollers 105 operably disposed inside roller frame 106, and are compressively adhered and flexed upwards as a double layer which is fixed in grip 107. The roller frame 106 is maintained in optimal position near the skin surface by holding and maintaining handle 108 level adjacent skin 104 by physical force, and pulling vertically on grip 107. In the example, the roller frame 106 can be further positionably stabilized by base plates 109, which may or may not be material parts of the device 101. The device 101, typically manufactured in polymer, may eventually but not necessarily be hollow.

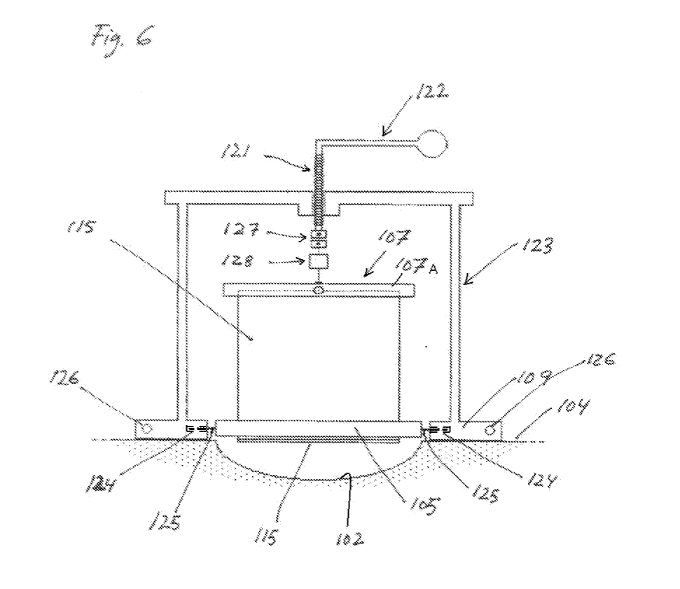

Vertical manual force or pull applied to upwards extension of flanges 103 by means of grip 107 is redirected by rollers 105 to pull medially on the skin 104 on either side (see arrows indicating force vectors). During this the vertical pull, rollers 105 are kept in position at the skin level by means of downwards pressure on handle 108. The forces acting upwards and downwards will thus neutralize each other partially. Both roller frame 106 and grip 107 are preferably but not necessarily fitted with hinges 112 to ease application and insertion of the layers 110 and 111 therethrough. Roller frame 106 is closed by means of a hook (see FIG. 5) and grip 107 is locked by screw 113 (see FIGS. 3 and 4). The wound 102, can be covered by polymer film 114 which is advantageously treated continuously for instance by use of a negative pressure operated pump according to the parent application.

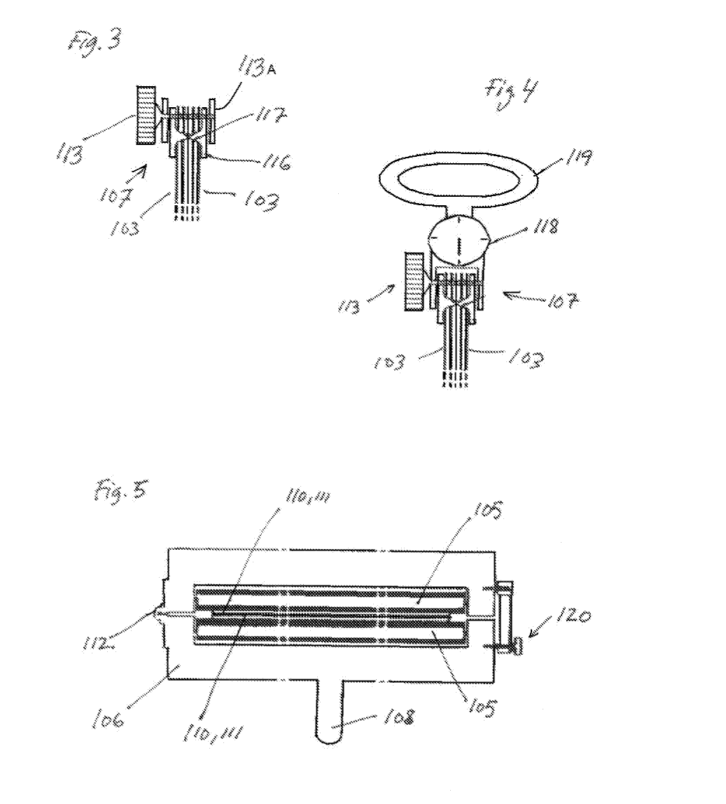

FIG. 2 shows a partial view similar to that of FIG. 1. Here, there is a single directionally stable adhesive layer 115 employed in opposing relation. Flanges 103 are constituted by a layer 115 being drawn together through rollers 105.

FIG. 3 shows a cross section of grip 107 comprising two rigid polymer or metal plates 116 fitted medially with tapered points 117 which aid to immobilize plates 116 to the vertical extensions of flanges 103 once screw 113 is turned through threaded surface of plate 113A. In FIG. 4 said grip 107 can be fitted with a traction force gauge 118 and handle 119.

FIG. 5 shows a schematic cross-sectional view of rollers 105 inside frame 106 during operation. Frame 106, kept immobile at the skin level by physical force applied to handle 108, can be opened about hinge 112 and is in the figure locked by means of hook 120. Frame 106 holds rollers 105 in compressing contact with double layer sheets 110 and 111 (or optionally layers 115) as the layers enter frame 106 below and from either side as grip 107 is pulled.

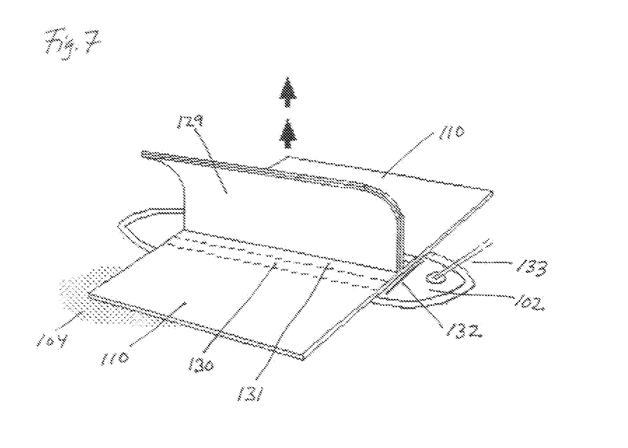

FIG. 6 shows a schematic clinical cross-sectional view of a traction device of an alternative design which is instead operated by means of screw 121 and lever 122. A stand 123 rests on base plates 109 on either side of wound 102. Base plates 109 each include a pair of coaxially aligned inwardly extending cylindrical holes 124 in which the axles 125 of rollers 105 are operably positioned. To accomplish the insertion of axles 125, the axles 125 may be formed with by way of spring loaded in roller 105 or alternatively the base 109 can be divided crosswise through the holes 124 and comprised of multiple parts which connect via a screw 126, for example. Traction polymer sheets 115, adhered adhesively to adjacent skin 104 pass medially between rollers 105, deviate vertically and are superiorly securely connected between each blade 107A of grip 107. Grip 107 is pulled vertically when lever 122 turns screw 121 above joint 127. A traction force gauge 128 can be interposed between grip 107 and a joint 127 allowing upwards rotatory movement according to the state of the art.

FIG. 7 shows a perspective view of ongoing passive traction according to the invention. Sufficient vertical flange 129 is left to give room for repeating traction at shorter intervals without reapplying a new set of flanges to the skin. Two such planned medial traction steps, 130 and 131 are shown. The original cover sheet protecting the adhesive side of the flange sheet 132 has been turned upside down and adhered to the otherwise exposed adhesive undersurface of traction-exposed film 110, preventing accidental adhesion when treating the underlying wound 102 by suctioning through tube 133. Prior to reactuating traction, the traction device is reapplied and flange sheet 132 removed, exposing the dependent adhesive flange surfaces. The traction is then engaged as indicated in one or two steps are suggested by the broken lines 130 and 131.

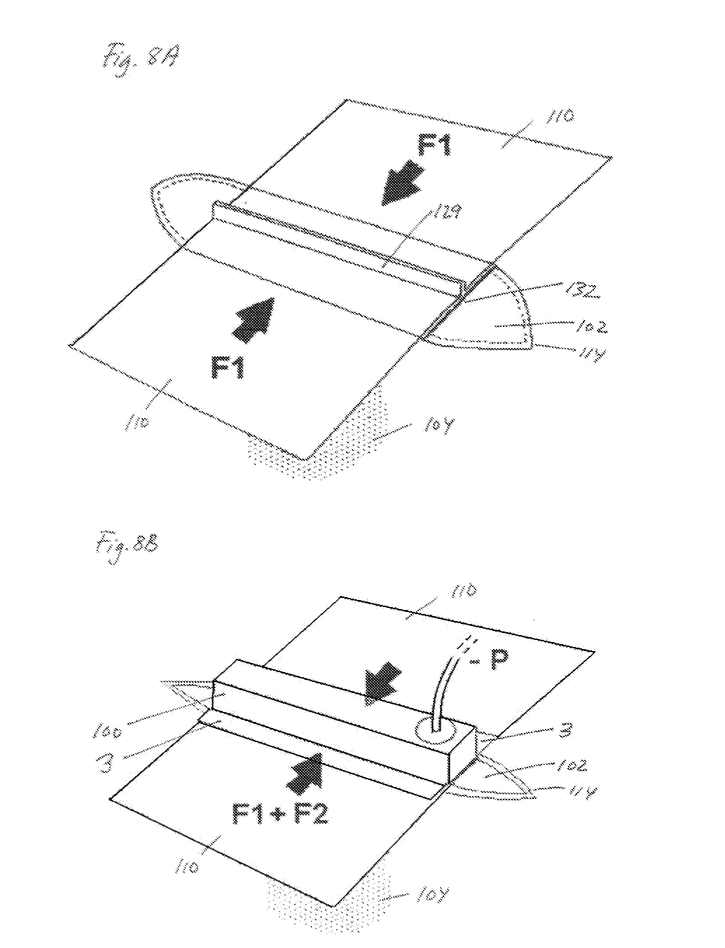

FIGS. 8A and 8B show schematic functional views of a clinical method for increasing medial traction force by combining manual and negative pressure techniques according to the present inventions. FIG. 8A shows an application according to the present invention whereby manual traction is first employed. The skin 104 is pulled medially by force F1. In FIG. 8B suction-operated actuator 100 has been adhered adhesively to underlying flanges according to FIG. 8A. The arrows indicate traction forces, of which F1 is mechanically and F2 vacuum actuated.

The above described embodiments are set forth by way of example and are not for the purpose of limiting the present invention. It will be readily apparent to those skilled in the art that obvious modifications, derivations and variations can be made to the embodiments without departing from the scope of the invention. Accordingly, the claims appended hereto should be read in their full scope including any such modifications, derivations and variations.

I claim:

1. A traction device, which includes:
an advancing member equipped with a zone through which a member is forcibly advanced,
a pair of cooperating flexible members, wherein each flexible member has at least one side thereof at least partially coated with an adhesive material, wherein said adhesive material of each said member is connected to opposing skin surrounding a wound such that a remaining portion of each member extends over and above the wound and wherein the remaining portions are connected through said zone of said advancing member in a manner to advance said remaining portions and draw the opposing surrounding skin toward each other in a manner to aid in closure of the wound.

2. The traction device of claim 1, wherein said advancing member includes a frame through which said remaining portions pass.

3. The traction device claim 2, wherein said frame is disposed medially over the wound adjacent the skin.

4. The traction device of claim 1, wherein said advancing member includes a pair of nips through which said remaining portions pass.

5. The traction device of claim 4, wherein said nips are disposed medially over the wound adjacent the skin.

6. The traction device of claim 1, which further includes a gripping member connectable to said remaining portions to aid in pulling said connected remaining portions.

7. The traction device of claim 6, which further includes a device connected to said gripping member for pnuematically pulling said remaining portions.

8. The traction device of claim 6, which further includes a device connected to said gripping member for mechanically pulling said remaining portions.

9. The traction device of claim 2, which further includes a gripping member connectable to said remaining portions to aid in pulling said connected remaining portions through said frame.

10. The traction device of claim 9, which further includes a one of a mechanical and pneumatic device connected to said gripping member for pulling said remaining portions through said frame.

11. The traction device of claim 4, further includes a gripping member connectable to said remaining portions to aid in pulling said connected remaining portions.

12. The traction device of claim 11, which further includes a one of a mechanical and pneumatic device connected to said gripping member for pulling said remaining portions through said nips.

13. The traction device of claim 1, which includes elastic, compressible open cell material and is airtightly connected to a negative pressure source.

14. The traction device of claim 13, whereupon negative pressure being exerted said material contracts medially and the force created by suction exerts medial traction on said portions of said members adhesively connected to the surrounding skin.

15. The traction device of claim 14, wherein said portions of said members adhesively connected to the surrounding skin have an inner, circumferential rim free from adhesive.

16. The traction device of claim 15, wherein said portions of said members adhesively connected to the surrounding skin which have an inner, circumferential rim free from adhesive and are transparent to allow visual assessment of the underlying skin with respect to viability.

17. The traction device of claim 15, wherein said portions of said members adhesively connected to the surrounding skin contain pores which allow evaporation of water.

18. The traction device of claim 1, wherein said members partially cover said would leaving access for applying a wound dressing from the sides.

19. The traction device of claim 1, wherein said members include one of active substances distributed in fiber, open cell or gel material.

20. The traction device of claim 1, wherein said members include one of an occlusively applied typically open cell, pore and fiber dressing with an access port used for negative pressure wound treatment.

21. The traction device of claim 20, which includes a therapeutic fluid supply to said dressing.

22. The traction device of claim 13, wherein said negative pressure is equipped with one of a measuring gauge, a mechanism to adjust and maintain the pressure according to clinical demand, an alarm to warn of air leak in the system, a computer controller, portability, and automation to provide continuous, intermittent or cyclic application of skin traction according to the invention may enhance blood flow and stimulate tissue expansion.

23. The traction device of claim 1, wherein said portions of said members adhesively connected to the surrounding skin flange include perforated mesh with pores permitting excess fluid to be drained from underlying cavity or the wound.

24. A method for applying traction to tissues adjacent a wound, which includes the steps of:
(a) positioning a contractible actuator adjacent a wound, wherein said actuator has portions adhesively connected to surrounding skin of the wound; and
(b) actuating the actuator to draw said portions medially inwardly to effect closure of the wound.

25. The method of claim 24 which includes providing negative pressure on the wound.
